(19) United States
(12) Patent Application Publication
(10) Pub. No.: US 2012/0226363 A1
(43) Pub. Date: Sep. 6, 2012

(54) METHOD FOR MANUFACTURING A DEVICE FOR REGENERATING BIOLOGICAL TISSUES

(76) Inventor: Alessandro Sannino, Lecce (IT)

(21) Appl. No.: 13/503,134

(22) PCT Filed: Oct. 7, 2010
(86) PCT No.: PCT/EP2010/065046

§ 371 Date: May 8, 2012

(30) Foreign Application Priority Data
Oct. 20, 2009 (IT) MI2009A001807

(51) Int. Cl. A61F 2/02; B29C 35/16

(52) U.S. Cl. 623/23.72; 264/28; 425/384

(57) ABSTRACT

A device for regenerating biological tissues, particularly for regenerating tissues of the peripheral nervous system, the respective manufacturing method and the instrument used in the method, the regeneration device comprising a hollow tubular structure based on biocompatible material and having a structural porosity in which the pores are oriented substantially radially with respect to its longitudinal axis so as to minimize the formation of scar tissue around the damaged site and allow the growth of the biological tissue inside the pores and inside the duct defined by the hollow tubular structure and facilitate the transport of nutrients.

### TECHNICAL FIELD

The present invention relates to a device for regenerating biological tissues, particularly for regenerating tissues of the peripheral nervous system, to the respective manufacturing method, and to the instrument used in said method.

### BACKGROUND ART

In recent years, regenerative medicine has become increasingly widespread as a therapeutic method for treating several types of injury. In this modern approach, for example, one attempts to close a skin wound by promoting the synthesis of scar tissue.

In particular, in induced regeneration, a bioactive structure is arranged in the wound, modifying the original mechanism of healing, or repair, inducing regeneration of the physiological tissue.

In this process, an essential role is entrusted to regeneration devices known in the technical jargon as “scaffold”, which act both as physical supports and guides for the growth of the tissue and as regulators of cellular function, because they provide the adapted stimuli for tissue regrowth.

Scaffolds with appropriate composition, structural, mechanical and degradation characteristics can thus allow a regenerative healing process.

As regards lesions to nerves of the peripheral nervous system, the technique of tublation is becoming established as being particularly effective and consists substantially in using a tubular structure to induce regeneration of the lost nerve ending until the distal and proximal ends of a severed peripheral nerve are reconnected.

The presence of a connection between the two severed ends is a first essential factor in order to induce regeneration of the injured nerve.

However, it has been noted that the microstructural, mechanical and composition characteristics of the tubular structure proper and of any material inserted in the cavity of the tubular structure affect significantly the quality of the regeneration.

Besides providing a support to the direction of growth of the axons from the proximal ending to the distal ending, an ideal tubular structure in fact should protect the injured site from the infiltration of surrounding tissues and, at the same time, maintain a certain level of porosity.

In particular, the tubular structure should allow the spreading of cytokinesis and metabolites through the wall of the tube and affect the migration and organization of myofibroblasts, which are responsible for the unwanted synthesis of scar tissue.

The tubular structure should provide, moreover, an adequate mechanical force and flexibility to support the regeneration of the nerve fibers and should be biocompatible and biodegradable.

Over time, the quality of nerve regeneration has been improved with the control and selection of parameters related to the tube, such as the length and diameter of the tube, the microgeometry of the hollow inner surface, the porosity of the wall of the tube, hydrophilicity and permeability.

Tubular structures made of collagen and provided with a porous wall, with a random pore microstructure that ensures permeability to proteins and to cells, allows to obtain high-quality regeneration of the peripheral nerve.

Several techniques are known for the production of structures with porous walls. However, such techniques suffer drawbacks, including: the limitation of the size of the molds, the size, structure and number of the pores, the need to use a complex tubular mold and, last but not least, the complexity of many processes required to provide the structure.

In particular, the use of complex molds requires careful handling of the product during all the steps of production and also during removal of the samples from the mold.

A significant improvement in the provision of scaffolds is shown in US Patent Application 2008/0102438 which discloses a method for producing collagen tubes in which a collagen suspension is inserted in a mold until it fills it.

The mold is placed under rotation about its own axis and then porosity is created by immobilizing part of the components that constitute the suspension and subsequently removing them.

In particular, in the case of water-based collagen suspension, in order to immobilize and subsequently remove the components one uses a process in two steps: first the sedimentation of the solid phase (i.e., the collagen) in the aqueous solution, controlled by parameters set by the operator; then a process known in the jargon as “freeze-drying”.

With the method disclosed in this patent, tubular scaffolds made of collagen are obtained with a pore size gradient that decreases along the radius of the tube, a pore distribution that is oriented along the radius, and an external surface that is permeable to proteins and cells.

This method makes it possible to control the geometry and the porosity of the tubular structure easily and precisely.

Despite the good results obtained, the method and the product described in the above-mentioned patent are not completely devoid of drawbacks.

### DISCLOSURE OF THE INVENTION

The aim of the present invention is to provide a device for regenerating biological tissues and the respective manufacturing method, particularly for regenerating tissues of the peripheral nervous system, which improve the results that can be obtained with the background art.

Within this aim, an object of the present invention is to provide a regeneration device that is capable of protecting the site of the implant from the infiltration of external tissue but remains permeable to cells from the inside outward.

Another object of the invention is to provide a regeneration device with a smoother inner surface, so as to optimize the regrowth of an injured nerve.

Still another object of the invention is to devise a method for manufacturing a regeneration device that minimizes the quantity of collagen or, more generally, of biocompatible material to be used.

Another object of the invention is to provide a regeneration device that is highly reliable, relatively easy to provide and has competitive costs.

This aim and these and other objects that will become better apparent hereinafter are achieved by a device for regenerating biological tissues, characterized in that it comprises a hollow tubular structure based on biocompatible material having a structural porosity in which the pores are oriented substantially radially with respect to its longitudinal axis so as to allow the growth of the biological tissue inside said pores and inside the duct defined by said hollow tubular structure, and by the respective manufacturing method, characterized in that it comprises the following steps:

preparing an aqueous suspension of biocompatible material,

injecting said aqueous suspension in a mold that has an inner cavity having a substantially elongated shape along a predefined direction and a substantially circular transverse cross-section,

rotating said mold about a rotation axis for the sedimentation of said aqueous suspension on the lateral walls of said mold and the generation of a hollow tubular structure which is coaxial to said rotation axis,

immersing, in a bath of liquid nitrogen, said rotating mold containing said aqueous suspension along an immersion path that substantially coincides with said longitudinal axis for freezing said aqueous suspension,

sublimating said aqueous suspension contained in said mold,

extracting from said mold the device for regenerating biological tissues as obtained at the end of said sublimation step,

drying said regeneration device,

characterized in that during said injection step said inner cavity is filled only partially with said aqueous suspension.

Moreover, this aim and these and other objects that will become better apparent hereinafter are achieved by a device for regenerating biological tissues, characterized in that it comprises a hollow tubular structure based on biocompatible material having a structural porosity in which the pores are oriented substantially radially with respect to its longitudinal axis so as to allow growth of the biological tissue inside said pores and inside the duct defined by said hollow tubular structure, and by the respective manufacturing method, characterized in that it comprises the following steps:

preparing an aqueous suspension of biocompatible material,

injecting said aqueous suspension in a mold that has an inner cavity having a substantially elongated shape along a predetermined direction and a substantially circular transverse cross-section,

rotating said mold about a rotation axis for the sedimentation of said aqueous suspension on the lateral walls of said mold and the generation of a hollow tubular structure which is coaxial to said rotation axis,

immersing said rotating mold in a bath of liquid nitrogen along an immersion path that substantially coincides with said longitudinal axis for freezing said aqueous suspension,

sublimating said aqueous suspension contained in said mold,

extracting the device for regenerating biological tissues, particularly for regenerating tissues of the peripheral nervous system, obtained at the end of said sublimation step, from said mold,

drying said regeneration device in a dryer,

characterized in that said mold comprises, at its outer surface, an outer sheath made of a material with high thermal conductivity to improve the properties of heat exchange between the internal region formed by said inner cavity and said outer surface.

### BRIEF DESCRIPTION OF THE DRAWINGS

Further characteristics and advantages of the invention will become better apparent from the description of a preferred but not exclusive embodiment of the device for regenerating biological tissues and of the respective manufacturing method, according to the invention, illustrated by way of non-limiting example in the accompanying drawings, wherein:

FIG. 1 is a perspective view of a regeneration device according to the invention;

FIG. 2 is a flowchart of the method for manufacturing the regeneration device according to the invention;

FIG. 3 is a perspective view of the mold according to the invention;

FIG. 4 is a perspective view of a supporting structure, in which the mold is to be inserted, for rotation;

FIG. 5 is a detail view of a rotation apparatus during the rotation step;

FIG. 6 is a detail view of an apparatus during the freezing step.

### WAYS OF CARRYING OUT THE INVENTION

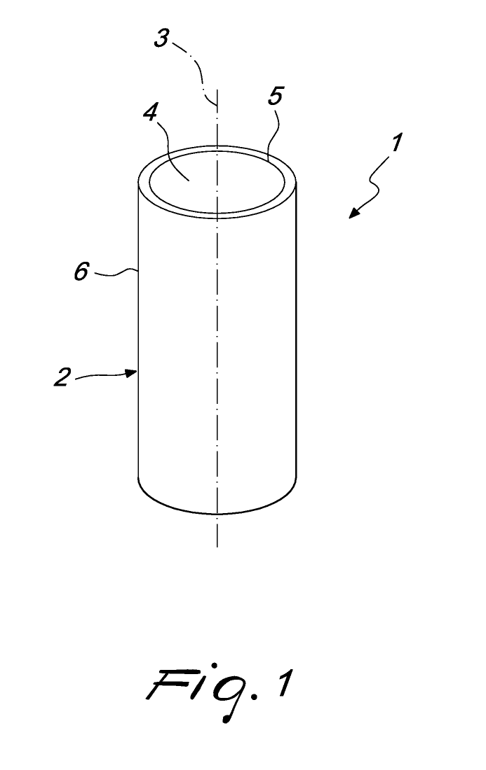

With reference to FIG. 1, the regeneration device according to the invention, generally designated by the reference numeral 1, comprises a hollow tubular structure 2, which is based on biocompatible material, such as for example collagen, and can be interposed between the two ends of the biological tissue to be regenerated.

The hollow tubular structure 2 has a structural porosity whose pores are oriented substantially radially with respect to its longitudinal axis 3 so as to allow the growth of the biological tissue inside said pores and inside the duct 4 formed by said hollow tubular structure 2.

The inner wall 5 of the hollow tubular structure 2 has a smaller volumetric solid fraction and therefore a lower relative density of collagen, and a greater average size of the pores so as to constitute a region that is permeable to the cells that are present inside the duct 4.

Conveniently, as mentioned earlier, in order to allow a preferential migration of the cells inside the duct 4 toward the outer wall 6, the pores of the inner wall 5 are oriented substantially radially with respect to the longitudinal axis 3.

Differently from the inner wall 5, the outer wall 6 is permeable to proteins and impermeable to external cells to protect the implantation site from the infiltration of external biological tissue.

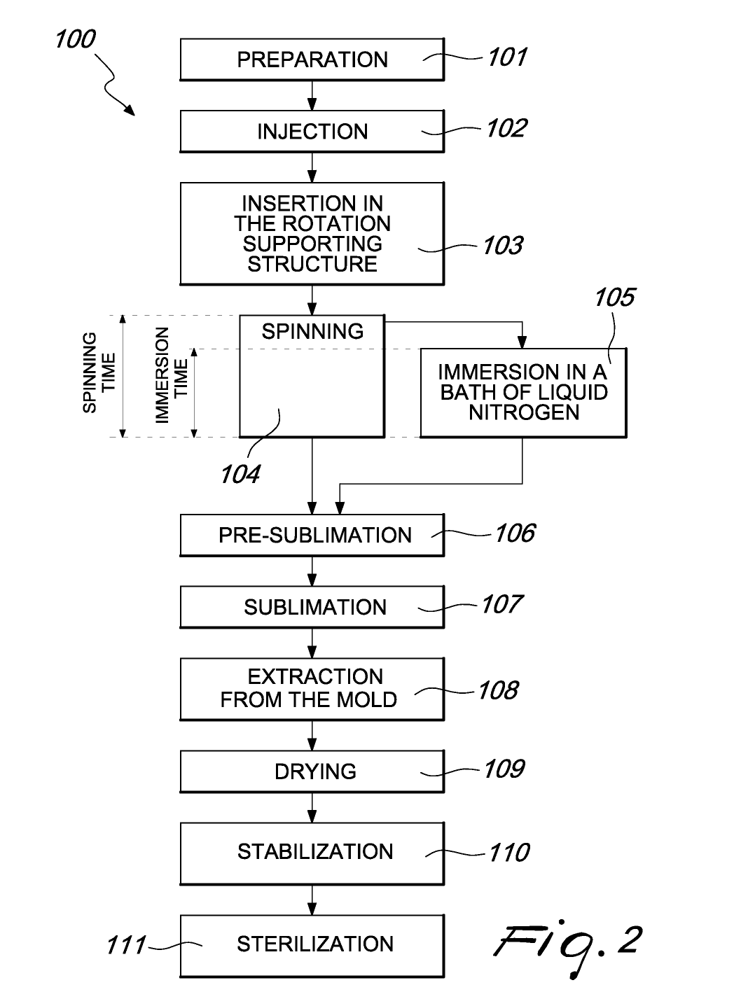

The manufacturing method 100 for manufacturing the regeneration device 1 according to the invention comprises the steps reported in the flowchart of FIG. 2.

More precisely, the manufacturing method 100 comprises a preparation step 101 in which the aqueous suspension 21 of biocompatible material is prepared. Preferably, this biocompatible material is Type I fibrillar collagen, which is derived, for example, from cattle hide, and contains a high solid content, for example equal to 3% by weight.

In the method 100, only the collagen is immersed in the aqueous solution, without the addition of other components.

However, the consistency and density of the collagen inside the liquid suspension can vary to produce a specific porous structure, which is necessary for a particular use, in the manners known to a person skilled in the art.

Advantageously, the preparation step 101 comprises a centrifugation of the aqueous suspension 21 for eliminating the air that is present in the aqueous suspension 21.

More particularly, the aqueous suspension 21 can be centrifuged, for example, for 12 minutes at 6000 rpm.

The aqueous suspension 21 is then maintained at a temperature of about 4° C. and, before use, it is left for a few hours at room temperature, comprised between 18° C. and 20° C., so as to reduce its viscosity and thus facilitate the subsequent injection step 102.

In the injection step 102, the aqueous suspension is injected into a mold 11 by means, for example, of a graduated pipette.

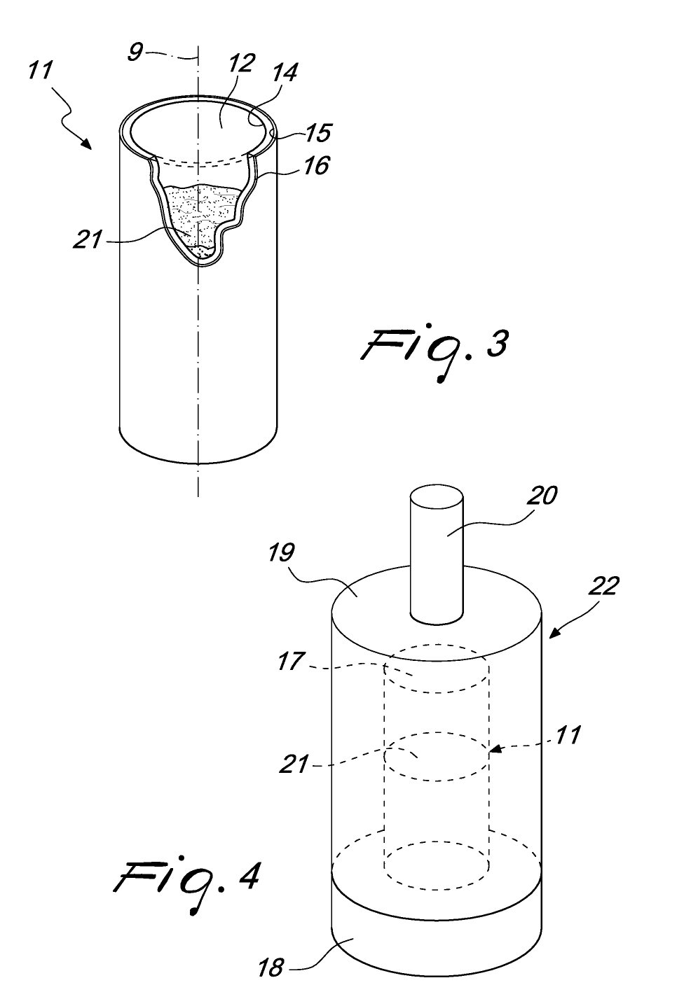

With reference to FIG. 3, the mold 11, which can be made of PVC (polyvinyl chloride) or silicone, defines an inner cavity 12 which is substantially elongated along a predetermined direction 9, which coincides with its longitudinal axis, and has a substantially circular transverse cross-section.

More precisely, the lateral surface 14 of the inner cavity 12 defines the shape of the outer wall 6 of the regeneration device 1 and can have dimensions, inside diameter and length, that can vary depending on the specific application.

A particularity of the invention consists in that the mold 11 can have, at its outer lateral surface 15, an outer sheath 16 made of a material with high thermal conductivity for improving the properties of heat exchange between the internal region defined by said inner cavity 12 and the outside environment.

Preferably, the outer sheath 16 is made of copper or other material having a similar thermal conductivity.

Another particularity of the invention consists in that the inner cavity 12 of the mold 11 is filled only partially and not completely with inner aqueous suspension 21. Preferably, substantially half of the available volume defined by the cavity 12 of the mold 11 can be filled with the aqueous suspension 21. In this manner, the quantity of biocompatible material, in particular of collagen, to be used in order to provide the regeneration device 1 is optimized and minimized, providing an inner wall of the scaffold that is particularly smooth, well-defined and symmetrical.

Subsequently, in step 103 the mold 11 provided with the outer sheath 16 is first closed at one end by a plug 17, for example made of plastics, and is then inserted in a cylindrical body 19, preferably made of copper or other material having a similar thermal conductivity. The lower end of the cylindrical body 19 has a threaded portion adapted to be screwed inside the complementarily threaded portion of a base 18, which also is made of copper or other material having a similar thermal conductivity.

The base 18 is coupled to the cylindrical body 19 so as to constitute the base for the latter and thus form a rotating support 22.

In this manner, the outer copper sheath 16 of the outer lateral surface 15 of the silicone mold 11 acts as a jacket between the silicone mold 11 and the cylindrical copper body 19; in particular, the function of the outer sheath 16 is to ensure good adhesion between the silicone mold 11 and the cylindrical copper body 19 and an optimum distribution of the heat, thus allowing an optimum distribution of the pores that will be formed.

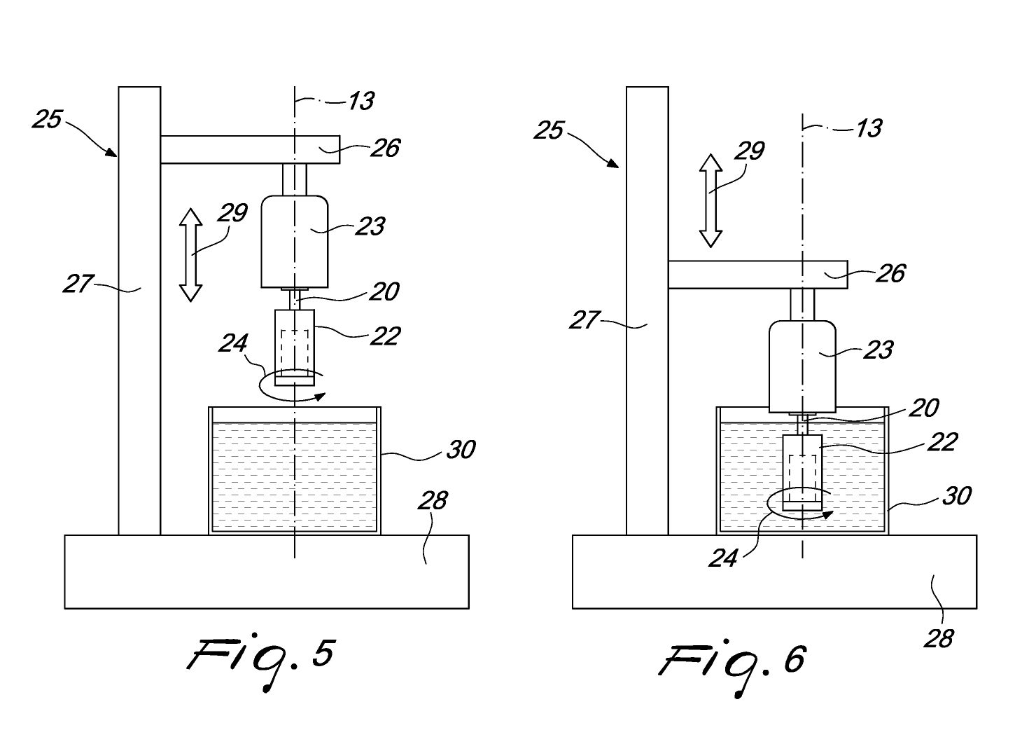

With reference to FIGS. 5 and 6, the rotating support 22, which comprises internally the mold 11 that contains the collagen suspension 21, is mounted on a motorized structure 23 by means of a rod 20, made for example of metallic material, which protrudes from the upper portion of the cylindrical body 19.

Such motorized structure is capable of producing a rotation 24, known in the technical jargon as “spinning”, about a rotation axis 13 that coincides substantially with said predetermined direction 9 of the mold 11.

In a particularly advantageous configuration, such as the one shown in FIGS. 5 and 6, the motorized structure 23 is fixed to a horizontal bar 26, which is coupled to a vertical pillar 27 arranged at right angles to a footing 28.

The coupling between the horizontal bar 26 and the pillar 27 is adapted to allow the movement 29 of the horizontal bar 26 along a direction that is substantially parallel to the rotation axis 13.

A bath of liquid nitrogen 30 is arranged on the upper face of the footing 28 in such a position that the rotation axis 13 of the mold crosses on the inside the volume formed by said bath of liquid nitrogen 30.

During the rotation step 104, the motorized structure 23 subjects the rotating support 22, with inside the mold 11 containing the aqueous collagen suspension 21, to the spinning 24 about the rotation axis 13 at a preset speed and time in order to cause a phenomenon of sedimentation of the aqueous collagen suspension 21 on the internal wall of the mold 11, thus generating a hollow tubular structure which is coaxial to the rotation axis 13.

In particular, by adjusting the rate of the spinning 24 of the rotating support 22 it is possible to adjust the inner diameter of the cavity 4 of the regeneration device 1.

The fact that the collagen is in an aqueous suspension and therefore the fact of having components of sufficient different density makes it possible to generate a hollow tubular structure that is coaxial to said rotation axis 13.

In the immersion step 105, the rotating support 22, with inside the mold 11 that contains the aqueous collagen suspension 21, still subjected to the spinning 24, is immersed in a bath of liquid nitrogen 30, by means of the movement 29 of lowering the horizontal bar 26.

In this step 105, the aqueous collagen suspension 21 that is contained in the mold 11 is frozen for a preset period of time, at the end of which the rotating support 22 is extracted from the bath of liquid nitrogen 30 and the spinning 24 is stopped.

Immersion in the bath of liquid nitrogen 30 causes the freezing of the aqueous collagen suspension 21, and more particularly, the aqueous component, by solidifying, forms ice crystals inside the hollow tubular structure obtained by the sedimentation of the collagen suspension 21 on the internal wall 5 of the mold 11.

Advantageously, the fact that the spinning 24 continues also during the immersion step 105 allows the creation of a hollow tubular structure having a structural porosity in which the pores are oriented substantially radially with respect to the rotation axis 13.

Once the rotating support 22 has been removed from the bath of liquid nitrogen 30, the mold 11 is extracted from the rotating support 22 and is introduced in a freeze-dryer, where the pre-sublimation step 106 occurs.

During step 106, the mold 11 is kept at a predefined temperature, preferably equal to −40° C., for a preset time equal to 1 hour.

Subsequently, still inside the freeze-dryer, the sublimation step 107 occurs in which first the inside pressure of the freeze-dryer is lowered to a preset value, preferably equal to 200 mTorr, keeping the temperature preferably equal to −40° C., and then, once such pressure value has been reached, the temperature inside the freeze-dryer rises to a preset value preferably equal to 0° C.

The mold 11 is kept at said temperature for a preset time, preferably equal to 17 hours, and then the inside temperature of the freeze-dryer is raised to a preset value, preferably equal to 20° C., and the previously obtained crystals are melted.

Subsequently, once air has been injected into the freeze-dryer for restoring the atmospheric pressure inside it, the mold 11 is removed from the freeze-dryer.

In the subsequent extraction step 108, the regeneration device 1 thus obtained is removed from the mold 11.

Finally, in step 109 the regeneration device 1 is arranged in a dryer in order to be dried.

In this manner, the aqueous component of the suspension is removed and the desired porous structure of the regeneration device 1, in which the pores are oriented substantially radially to its longitudinal axis 3, is obtained.

The longitudinal axis 3 of the regeneration device 1 thus obtained substantially coincides with the rotation axis 13.

As already mentioned, the inside diameter of the hollow tubular structure 2 of the regeneration device 1 and the gradient of the pores depend on the speed and time of the spinning 24.

The gradient in the number and size of the pores along the radius of the hollow tubular structure 2 are obtained as a result of the combined effect of sedimentation and of the heat transfer gradient.

Precise control of the temperature and pressure inside the freeze-dryer during steps 106 and 107 makes it possible to modulate the dimensions of the ice crystals that are generated inside the hollow tubular structure 2 following immersion in the liquid nitrogen bath 30. Modulating the size of the crystals makes it possible to intervene on the size of the pores downstream of the drying step 109.

The pore size needs very precise control to facilitate the migration of a specific type of cells, the myofibroblasts, so as to eliminate them from the site of the lesion.

As already mentioned, the outer surface 6 of the regeneration device 1 thus obtained has a higher relative density of collagen and a reduced average pore size, so as to be a region that is permeable to proteins and impermeable to cells.

Differently, the inner wall 5 of the regeneration device 1 has a smaller volumetric solid fraction and therefore a lower relative density of collagen, and a greater average size of the pores so as to constitute a cell-permeable region inside the cavity 4 of the regeneration device 1. Preferably, the regeneration device 1 thus obtained can undergo a stabilization step 110 with the purpose of reducing the degradation rate when implanted.

Stabilization step 110 occurs by means of a cross-linking treatment that acts on the density of the cross-linking bonds that exist among the macromolecules of the collagen.

More specifically, the procedure used can be DeHydroThermal Cross-Linking (DHT), which is a chemical cross-linking treatment that does not use cross-linking agents and in particular is performed in a vacuum oven for a period of time that varies from 24 to 48 hours at a temperature preferably equal to 121° C. and a pressure preferably equal to 100 mTorr.

Finally, advantageously, the regeneration device 1 undergoes a dry heat sterilization step 111, which makes it possible not to damage and degrade the structural integrity of the regeneration device 1. The sterilization treatment with dry heat (Dry-Heat Sterilization, DHS) is preferably performed in a vacuum oven in standard conditions, i.e., for a period of time preferably equal to 2 hours and at a temperature preferably equal to 160° C.

In practice it has been found that the method according to the invention fully achieves the intended aim, since it allows the provision of a regeneration device capable of facilitating the regrowth of biological tissue.

In particular, the regeneration device, having a hollow tubular structure, is capable of being interposed between two ends of the biological tissue to be regenerated, in particular between the two injured ends of the peripheral nerve.

Moreover, the fact that the tubular structure of the regeneration device is provided with a structural porosity in which the pores are oriented substantially radially with respect to its longitudinal axis allows the regrowth of the biological tissue inside said pores and inside the duct defined by the hollow tubular structure 2.

Moreover, the fact that the outer wall of the regeneration device is provided with a higher relative density of collagen and has a reduced average pore size makes it a region that is permeable to the proteins and impermeable to the cells that are present outside the device.

The fact is also not negligible that the inner wall of the regeneration device, having a lower relative density of collagen and a larger average pore size, makes it possible to constitute a region that is permeable to the cells that are present inside the duct formed by the hollow tubular structure 2.

Moreover, the fact that the pores of the inner wall are oriented in a direction that is substantially radial with respect to the longitudinal axis of the regeneration device allows a preferential cell migration from the duct in the direction of the outer wall of the hollow tubular structure.

Moreover, the fact that the mold is provided with an outer sheath made of a material with high thermal conductivity, such as for example copper, makes it possible to improve the adhesion between the outer cylinder made of copper and the mold made of silicon and allows an improvement of the thermal exchange qualities and heat distribution qualities between the internal region defined by the inner cavity of the mold and the external environment.

Moreover, the fact that the aqueous collagen suspension fills the mold only partially makes it possible to obtain, already during the rotation step, the sedimentation of the aqueous collagen suspension on the inner wall of the mold, generating a hollow tubular structure that is coaxial to the rotation axis. Moreover, this characteristic allows minimization and optimization of the quantity of collagen to be used for providing the regeneration device.

Moreover, the adjustment of the rate and time of rotation make it possible to control the inside diameter of the tube and the gradient of the pores.

Moreover, by combining the sedimentation and the gradient of heat transfer it is possible to obtain a gradient in the number and size of the pores along the radius of the hollow tubular structure of the regeneration device.

Moreover, precise control of the temperature and pressure during the steps that occur inside the freeze-dryer allows modulation of the size of the ice crystals generated during immersion in the bath of liquid nitrogen and, therefore, the size of the pores is modulated downstream of the drying.

Precise control of the size of the pores makes it possible to obtain a pore size of about 20 micrometers, which is optimum in the regeneration of the peripheral nerve because it promotes cell migration and the elimination of the myofibroblasts from the site of the lesion.

Moreover, there is the fact that the resulting supporting element undergoes a stabilization step capable of decreasing the degradation rate of the supporting element in vivo by increasing the density of the cross-linking bonds that exist between the macromolecules of the collagen.

Also, the supporting element undergoes a dry heat sterilization step, which makes it possible to avoid damage and degradation of the chemical and physical qualities of the supporting element.

Although the method according to the invention has been conceived in particular for the manufacturing of devices for regenerating biological tissues, particularly for regenerating tissues of the peripheral nervous system, with a tubular shape and radial porosity pattern, it may nonetheless be used and adapted, more generally, for manufacturing regeneration devices with other shapes and other porosity patterns.

The regeneration device and the corresponding manufacturing method, as well as the mold used, thus conceived, are susceptible of numerous modifications and variations, all of which are within the scope of the appended claims; all the details may further be replaced with other technically equivalent elements.

In practice, the materials used, as well as the dimensions, may be any according to requirements and to the state of the art.

The disclosures in Italian Patent Application No. MI2009A001807 from which this application claims priority are incorporated herein by reference.

Where technical features mentioned in any claim are followed by reference signs, those reference signs have been included for the sole purpose of increasing the intelligibility of the claims and accordingly, such reference signs do not have any limiting effect on the interpretation of each element identified by way of example by such reference signs.

1-15. (canceled)

16. A method for manufacturing a device for regenerating biological tissues, particularly for regenerating tissues of the peripheral nervous system, comprising the following steps:
preparing an aqueous suspension of biocompatible material,
injecting said aqueous suspension in a mold that has an internal cavity having a substantially elongated shape along a predefined direction and a substantially circular transverse cross-section,
rotating said mold about a rotation axis for the sedimentation of said aqueous suspension on the inner wall of said mold and the generation of a hollow tubular structure which is coaxial to said rotation axis,
immersing, in a bath of liquid nitrogen, said rotating mold containing said aqueous suspension along an immersion path that substantially coincides with said longitudinal axis for freezing said aqueous suspension,
sublimating said aqueous suspension contained in said mold,
extracting from said mold the device for regenerating biological tissues as obtained at the end of said sublimation step,
drying said regeneration device,
characterized in that during said injection step said inner cavity is filled only partially with said aqueous suspension.

17. The manufacturing method according to claim 16, wherein said mold comprises, at its outer surface, an outer sheath made of a material with high thermal conductivity to improve the properties of heat exchange between the internal region formed by said inner cavity and said outer surface.

18. A method for manufacturing a device for regenerating biological tissues, particularly for regenerating tissues of the peripheral nervous system, comprising the following steps:
preparing an aqueous suspension of biocompatible material,
injecting said aqueous suspension in a mold that has an inner cavity having a substantially elongated shape along said predefined direction and a substantially circular transverse cross-section,
rotating said mold about a rotation axis for the sedimentation of said aqueous suspension on the inner wall of said mold and the generation of a hollow tubular structure which is coaxial to said rotation axis,
immersing said rotating mold containing said aqueous suspension in a bath of liquid nitrogen along an immersion path that substantially coincides with said longitudinal axis for freezing said aqueous suspension,
sublimating said aqueous suspension contained in said mold,
extracting from said mold the device for regenerating biological tissues, particularly for regenerating tissues of the peripheral nervous system, obtained at the end of said sublimation step,
drying said regeneration device in a dryer,
wherein said mold comprises, at its outer surface, an outer sheath made of a material with high thermal conductivity to improve the properties of heat exchange between the internal region formed by said inner cavity and said outer surface.

19. The manufacturing method according to claim 18, wherein in said injection step said inner cavity is partially filled with said aqueous suspension.

20. The manufacturing method according to claim 17, wherein said preparation step comprises a centrifugation of said aqueous suspension to eliminate the air that is present in said aqueous suspension.

21. The manufacturing method according to claim 17, wherein said preparation step comprises keeping said centrifuged aqueous suspension at ambient temperature for a preset time in order to reduce its viscosity.

22. The manufacturing method according to claim 17, wherein said outer sheath is made of copper.

23. The manufacturing method according to claim 17, further comprising a step of pre-sublimation of said aqueous suspension contained in said mold at a preset temperature, preferably equal to −40° C., and for a preset time, preferably equal to 1 hour, said pre-sublimation step being performed between said immersion step and said sublimation step.

24. The manufacturing method according to claim 17, wherein said sublimation step comprises:
lowering the inside pressure of a freeze-dryer containing said mold to a preset value, preferably equal to 200 mTorr, holding said temperature preferably equal to −40° C.,
raising the inside temperature of said freeze-dryer to a preset value, preferably equal to 0° C.,
holding said inside temperature for a preset time, preferably equal to 17 hours,
raising the inside temperature of said freeze-dryer to a preset value, preferably equal to 20° C.,
injecting air into said freeze-dryer and restoring atmospheric pressure inside said freeze-dryer.

25. The manufacturing method according to claim 17, further comprising a step for stabilizing said regeneration device so as to reduce the degradation rate of said regeneration device in vivo by means of a cross-linking treatment, said stabilization step being performed after said extraction step.

26. The manufacturing method according to claim 17, further comprising a step of sterilization of said regeneration device with dry heat.

27. A mold for providing a device for regenerating biological tissues, particularly for regenerating tissues of the peripheral nervous system, with a manufacturing method according to claim 16, having an inner cavity that is substantially elongated along a preset direction and has a substantially circular transverse cross-section, comprising, at its outer surface, an outer sheath made of a material with high thermal conductivity to improve the properties of heat exchange between the internal region formed by said inner cavity and said outer surface.

28. A device for regenerating biological tissues, particularly for regenerating tissues that belong to the peripheral nervous system, obtainable by a manufacturing method according to claim 17, comprising a hollow tubular structure based on biocompatible material having a structural porosity in which the pores are oriented substantially radially with respect to its longitudinal axis so as to allow the growth of the biological tissue inside said pores and inside the duct defined by said hollow tubular structure.

29. The regeneration device according to claim 28, wherein said hollow tubular structure comprises an inner wall which is permeable to the cells that are inside said duct.

30. The regeneration device according to claim 28, wherein said hollow tubular structure comprises an outer wall, which is permeable to proteins and impermeable to the cells that are outside said outer wall, so as to protect the implantation site against the infiltration of external biological tissue.
